(19) United States
(12) Patent Application Publication
(10) Pub. No.: US 2015/0127065 A1
(43) Pub. Date: May 7, 2015

(54) PERCUTANEOUS NEUROSTIMULATOR

(71) Applicant: Mario PASTORELLI, Grosseto(GR) (IT)

(72) Inventor: Mario PASTORELLI, Grosseto(GR) (IT)

(21) Appl. No.: 14/528,721

(22) Filed: Oct. 30, 2014

Related U.S. Application Data
(63) Continuation-in-part of application No. 13/731,041, filed on Dec. 30, 2012, now abandoned.
Application No. 13/731,041 is a continuation-in-part of application No. PCT/IT2011/000225, filed on Jun. 30, 2011.

(30) Foreign Application Priority Data
Jun. 30, 2010 (IT) RM2010A000354

(51) Int. Cl. A61N 1/36; A61N 1/378; A61N 1/04

(52) U.S. Cl. CPC A61N 1/36014; A61N 1/0484; A61N 1/378
USPC 607/59; 607/148; 607/144

(57) ABSTRACT

The present invention concerns a percutaneous neurostimulator (11) consisting of a printed circuit board, the main elements of which are a microcontroller (14) and two electrodes having opposed polarity, and operating according to an intensity of about 1 μA and a frequency or a combination of sequential frequencies chosen amongst eight possible frequencies: 8 Hz, 12 Hz, 16 Hz, 20 Hz, 24 Hz, 28 Hz, 32 Hz, 36 Hz.

This application is a continuation-in-part of application Ser. No. 13/731,041 filed Dec. 30, 2012 and a continuation-in-part of PCT/IT2011/000225 filed Jun. 30, 2011.

The present invention concerns a neurostimulator and more in particular a percutaneous neurostimulator for the correction of postural alterations of ascending nature starting from the feet and for the treatment of degenerative and inflammatory diseases of large and small joints.

The invention relates to the field of the correction of postural alterations, both static and dynamic, in subjects presenting alterations of posture starting from the feet and also to degenerative and inflammatory diseases of large and small joints (shoulder-elbow-wrist-hip-knee etc.). The more frequently and successfully treated diseases are scoliosis, headaches, TMJ disorders, knee pain, hip pain with initial structural problems, back pain, groin, injury, neurological type Parkinson's disease, brain and vascular injury, multiple sclerosis, the conflict of the shoulder, epicondylitis, de Quervain's disease, the trigger finger.

At present, in order to correct postural alterations starting from the feet, stimulation techniques have been used for foreign-proprioceptors of the foot making use of rigid components, such as insoles made of cork. For the purposes of the desired result, the height of such insoles, varying from 1 to 3 mm, is particularly important.

These activators, according to the technique of the French neurophysiologist D. R. J. Bourdiol, are positioned behind the metatarsal phalangeal joints (retrocapitate bar) or on the inside and sometimes outside of the calcaneus (heel wedges).

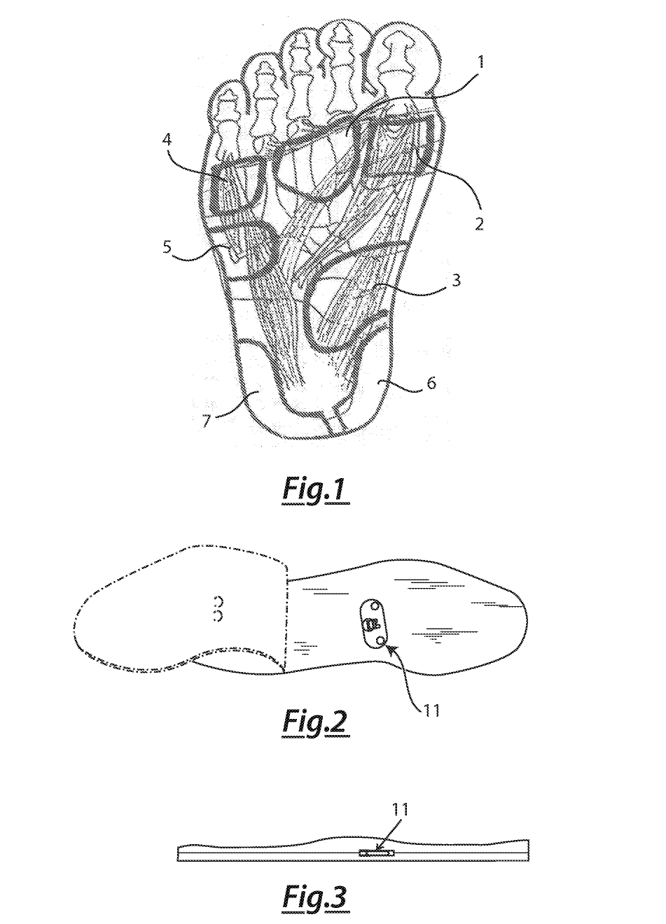

According to the technique based on applied kinesiology, the seats of stimulation of the foot are seven, i.e., by making reference to FIG. 1: hallux abductor (fx oblique and fx transverse) 1, short flexor of hallux 2, hallux abductor 3, short flexor of the fifth toe, 4, fifth toe abductor 5, medial or inner wedge 6, external or lateral wedge 7.

Other techniques of non mechanical neurological stimulation of the sole are Bricot's magnet and the bioresonance activated gel of Prof. Ricciardi.

In particular, Bricot's magnet is a non specific stimulus identical for all and with a time duration rather uncertain.

The activated gel, instead, works only for a few minutes, must then be spread under the foot several times a day.

In this context is integrated the solution according to the present invention, which aims to provide a device through which the neurological stimulation is made more accurate and scientifically measurable and can affect afferent stimuli to the reticular substance on the neuromuscular control joints, the spine and gait.

The applicant for the present invention in fact surprisingly discovered that the feet (and to some extent, medium and large joints like the shoulder, trapezium-metacarpal joint of the hands and wrists) are proper sense organs of posture. The applicant for the present invention also discovered that the possible neurological stimuli that can affect the afferent reticular substance for the neuromuscular control on the joints, the spine and gait that are actually effective are included in a finite number of predetermined values corresponding to frequencies: 8 Hz, 12 Hz, 16 Hz, 20 Hz, 24 Hz, 28 Hz, 32 Hz, 36 Hz.

Moreover, the applicant for the present invention also found that, although a specific ascending postural problem of breech type can be solved by application of a stimulus of a specific frequency, chosen amongst the frequencies: 8 Hz, 12 Hz, 16 Hz, 20 Hz, 24 Hz, 28 Hz, 32 Hz, 36 Hz, it is also true that the application of stimuli of a different frequency, always chosen from among those listed, has no effect, and nor has a negative effect.

For the rest, it is known that the feet respond to specific stimuli that can be mechanical (as in the case of insoles kinesiology) or neurological and non-specific stimuli such as activated gels and the magnet.

The present invention therefore aims to propose a percutaneous stimulation device that allows to exploit these discoveries.

The aim of the present invention is therefore to create a percutaneous neurostimulator device, to be applied to large and medium-sized joints, particularly the sole, the palm of the hand or wrist, capable of delivering a suitable current density, according to appropriate frequencies.

A further aim of the invention is that said device can be realised with substantially limited costs, both in terms of production costs and manufacturing costs.

Last but not least purpose of the invention is to realise a device which is substantially simple, safe and reliable.

It is therefore a specific object of the present invention a percutaneous neurostimulator constituted by a printed circuit board, the main elements of which are a microcontroller and two electrodes having opposed polarity, characterised in that it operates according to a frequency or a combination of sequential frequencies chosen amongst eight possible frequencies: 8 Hz, 12 Hz, 16 Hz, 20 Hz, 24Hz, 28 Hz, 32 Hz, 36 Hz.

Preferably, the percutaneous neurostimulator of the present invention further comprises a switch, which allows for changing the applied frequency choosing amongst the eight possible frequencies, starting from the lowest frequency and arriving up to the highest repeatedly with a change for every second.

Moreover, according to the invention, said percutaneous neurostimulator can further comprises an activation sensor can be powered alternatively with a battery, an accumulator, a piezoelectric generator.

Finally, always according to the present invention, said percutaneous neurostimulator can be applied alternatively not in direct contact with the skin, and in particular can be integrated within the sole of a shoe, within a tailor-made arch support, within elastic cuffs, or can be applied directly on the skin.

The present invention will now be described, for illustrative non limitative purposes, according to its preferred embodiments, with particular reference to some illustrative examples and the figures of the attached drawings, in which:

FIG. 1 shows the sole of a human foot and indicates the main muscles and the different stimulation areas,

FIG. 2 shows a top view of a footwear insole incorporating a percutaneous neurostimulator according to the present invention,

FIG. 3 shows a lateral view of the footwear insole and percutaneous neurostimulator of FIG. 2,

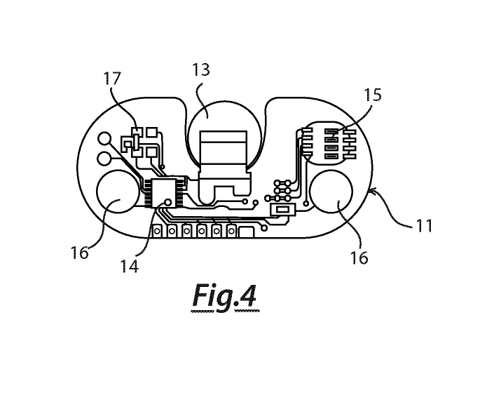

FIG. 4 shows a top view of a percutaneous neurostimulator according to the present invention.

The present invention relates to a percutaneous neurostimulator to be inserted in the footwear insole, in tailor-made plantars, in elastic cuffs, or to be applied directly on the skin, in correspondence of the acupuncture command points. The attached figures refer in particular, and for the purpose of example, the case where the percutaneous neurostimulator is applied to a footwear insole.

With reference to FIGS. 2 and 3, in which the numeral 10 is used to indicate a footwear insole and the numeral 11 indicates a percutaneous neurostimulator of the sole according to the present invention, the percutaneous neurostimulator 11 is located at the medial arch, with the electrodes 16 facing the outer hollow part. The assembly constituted by the insole and the stimulator integrated in it can be conveniently covered with a coating layer 12 (shown here partially lifted). In fact, it is not necessary that the electrodes are in direct contact with skin.

The size of the neurostimulator must be as reduced as possible, with rounded edges to reduce the risk of injury to the sole, to reduce the volume of the neurostimulator and to reduce the risk of system failure, with particular reference to the battery 13 and the electronic circuits.

The fundamental technical aspects the percutaneous neurostimulator 11 must consider are:
the intensity of current required,
frequencies applied,
the applied pulse width,
the device thickness and size, and
the battery life.

In particular, with reference to FIG. 4, the percutaneous neurostimulator 11 of the sole of a foot according to the present invention is constituted by a printed circuit board, powered by a 3V battery 13, the main elements of which are a microcontroller 14, a switch 15, two electrodes 16 and an activation sensor 17.

An important difference of the percutaneous neurostimulator 11 of the sole of a foot according to the present invention is the applied intensity. In fact, an intensity in the range about 1 μA is applied according to the present invention in order to condition the central nervous system, while on the contrary much higher intensities are applied by the neurostimulators according to the prior art, with the result that conditioning is limited to a local area surrounding the application point.

According to a possible embodiment, the microcontroller 14 operates with an intensity of 1 μA, a frequency of 4 MHz, by which it is possible to plan a precise impulse width of 50 microseconds. Due to the use of current, an activation sensor is needed, that switches the switch 15 in the rest position.

Alternatively, the microcontroller 14 operates with a clock frequency (32 kHz) and for this reason needs a smaller amount of current. It is possible to give up the activation sensor and the relative stop action. By means of a minimum clock, only a pulse width of 61-122 microseconds can be obtained.

Each electrostimulator contains a switch 15 (DPI switch) by which it is possible to control the applied frequency choosing amongst eight possible device frequencies, corresponding to those that proved to be actually effective for stimulation purposes, i.e. the eight possible frequencies: 8 Hz, 12 Hz, 16 Hz, 20 Hz, 24 Hz, 28 Hz, 32 Hz, 36 Hz. Instead of the switch it is also possible to set a fixed frequency, which can be chosen when programming, choosing amongst the eight possible frequencies: 8 Hz, 12 Hz, 16 Hz, 20 Hz, 24 Hz, 28 Hz, 32 Hz, 36 Hz, with a change for every second.

It is also possible to provide for the electrostimulator 11 being realised by replacing the battery with an accumulator, which can be charged by means of a recharge device through the contact points 16, or with a piezoelectric generator for recharging.

In particular, the accumulator, being rigid, can be placed in a central position and can be connected to two flexible fins where the positive and negative poles are housed for the application of the electrostatic field. All is then connected by two electric cables to a piezoelectric plate arranged below the plantar in correspondence of the forefoot.

A possible alternative power system consists in a thermal system, comprising two little plate of steel, in a different position, generating an electric field due to the different temperature the plates have depending of the different position.

A further alternative power system makes use on a sun battery system, to be applied to the wrist, again with an accumulator to allow the treatment even in absence of light.

Until today about 60 patients have been subjected to a treatment with the percutaneous neurostimulator of the present invention, said patients having difference problems going from osteoarthritis of the hip to muscular weakness in the pelvic girdle, or moreover low back pain, neck pain, groin and knee pain due to tendinitis, most of them having ascending postural problems starting from the feet. All the people subsequently visited had reacted positively to the application of the percutaneous neurostimulator of the present invention, in reason of the complete disappearance or at least of the evident improvement of the pain and functional symptoms. Also the baropodometric analysis showed a balancing of the static load, and muscle tests showed a return to normal values and symmetric.

Subsequent analysis after one, two or three months from application confirmed the improvement obtained soon after the application of the device.

Also patient with neurological lesions like Parkinson's, brain or vascular injuries, multiple sclerosis were treated with the percutaneous neurostimulator of the present invention, obtaining very interesting results.

In some cases, the percutaneous neurostimulator of the present invention was applied on the large and small joints like the shoulders, trapeziometacarpal joint of the hands, with encouraging results.

In the following some clinical cases are presented, treated with the neurostimulator of the invention.

In following examples 1-3, the chosen frequencies for treating the different patients were decided after applied kinesiology muscle testing, after years of evaluation on patients having ascending postural problems starting from the feet. In the following examples 4-8, on the contrary, a neurostimulator was used having a variable frequency of 8 Hz-12 Hz-16 Hz-20 Hz-24 Hz-28 Hz-32 Hz and 36 Hz changing every second, which does not require to set any specific frequency.

It was possible to verify that, in the frequency range comprised between 8 and 36 Hz, all the patients forming object of study responded positively.

### Application Example 1

The percutaneous neurostimulator of the sole of the present invention was applied to a 60 years old patient who presented the following symptoms:
impossibility of practising a sport activity.

Further, the kinesiological examination revealed the presence of a weak left side tensor and a weak rectus femoris. Thanks to the applied kinesiology tests it was possible to determine that the main postural problem depended from the right foot.

Hence, the neurostimulator according to the present invention was applied only to the right foot, setting an intensity of 1 μA and a fixed frequency of 8 Hz.

Subsequent weekly examinations revealed:
a clear reduction of pain after one week;
the disappearing of pain after three weeks.

Two months after the application the problems initially suffered by the patient were completely disappeared, and made it possible to start a sport activity.

### Application Example 2

A 25 years old patient suffering from knee pain, difficulty standing and and consequently working, who had previously followed a FANS therapy with topical and OS application without any positive result, was subjected to a postural kinesiology examination that allowed for diagnose a primary structural problem due to a weak left side tensor and a weak left ileus psoas. Moreover, the right foot shower a postural disturbance.

The applied therapy provided for the use of an anatomic insole in combination with the neurostimulator of the present invention, set at an intensity of 1 μA and a fixed frequency of 36 Hz.

One month after a clear improvement of knee pain and function was observed. The structural problem that had previously revealed did not result anymore if measured while the patient was using the neurostimulator of the invention, but still resulted without.

### Application Example 3

A 59 years old suffered pain and lameness due to an anatomic damage to the left hip joint (ascertained with x-Rays and RMN). Clinically a functional limitation was detected of the intra and extra rotation of the left hip of more than 50%, with atrophy of the left thigh and left buttock. Moreover, all the muscles of the hip joints (TFL, ileus psoas, gluteus medius, rectus femoris) resulted to be weak.

By a kinesiological examination it was possible to ascertain the presence of an ascending postural problem starting from both feet.

Anatomic insoles incorporating a neurostimulator according to the present invention set at an intensity of 1 μA and a frequency of 20 and 24 Hz were applied to the patient.

It was possible to observe an immediate improvement of the functions of the left hip and of the strength of the muscles examined.

Four months after, the patient said he felt good, he could walk without any pain, but only while wearing insoles incorporating the device of the invention.

### Application Example 4

Beside neurostimulators at fixed frequency set according to the specific needs of the patient, it was possible to verify the applicability of neurostimulators with variable frequencies of 8 Hz-12 Hz-16 Hz-20 Hz-24 Hz-28 Hz- 32 Hz and 36 Hz changing every second, controlled by a microprocessor. This kind of neurostimulators resulted to be particularly convenient because it allows for the use of the neurostimulator also for people that do not know any particular semiotic techniques.

The observed cases were all very positive, for hip pain, trigger finger, shoulder conflict.

This kind of neurostimulator was applied to a 78 years old patient, suffering from right hip joint pain since four years before.

Soon after the application of the neurostimulator according to this second embodiment of the present invention, the subject showed a 50% reduction of pain. Further, the subject completely solved his problem after 3 months using the neurostimulator of the present invention.

### Application Example 5

A 57 years old patient suffering left hip pain and disabling low back pain since 40 days before used the time variable frequency neurostimulator of the present invention. The patient showed a clear improvement at first control after 7 days, and further the total disappearance of pain after one month of treatment.

### Application Example 6

A 72 years old patient, subjected to surgery for a coxosteoarthritis 5 years before, suffered for an important pain to the right hip and an evident reduction of functionality. By using the time variable frequency neurostimulator of the present invention, the patient showed a clear reduction of pain and improvement of function after 10 days and a complete overcoming of its problems after three months.

### Application Example 7

### Epicondylitis

A baseball professional player, for about three months suffering from right elbow pain and consequently unable to play despite physiotherapy treatment, was applied the time variable frequency neurostimulator of the present invention on the ispilateral wrist. The day following the application the functional impotence ceased, and three days after the patient started to train again.

### Application Example 8

A patient suffering from epicondylitis with difficulty to work since about four months before, used a bracelet incorporating the time variable frequency neurostimulator of the present invention. The patient recovered the full functionality of the limb from the very subsequent day of the day of the application.

The present invention was described for illustrative, non limitative purposes, according to its preferred embodiments, but it has to be understood that variations and/or modifications can be made by the skilled in the art without escaping the corresponding scope of protection, as defined by the enclosed claims.

1. A percutaneous neurostimulator (11), wherein said percutaneous neurostimulator (11) consists of a printed circuit board, the main elements of which are a microcontroller (14) and two electrodes having opposed polarity, operating according to an intensity of about 1 μA and a frequency or a combination of sequential frequencies chosen amongst eight possible frequencies: 8 Hz, 12 Hz, 16 Hz, 20 Hz, 24 Hz, 28 Hz, 32 Hz, 36 Hz.

2. The percutaneous neurostimulator (11) of claim 1, wherein it further comprises a switch (15).

3. The percutaneous neurostimulator (11) of claim 2, wherein said switch (15) allows for changing the applied frequency choosing amongst the eight possible frequencies.

4. The percutaneous neurostimulator (11) of claim 3, wherein its frequency changes starting from the lowest frequency and arriving to the highest frequency repeatedly with a change for every second.

5. The percutaneous neurostimulator (11) according of claim 1, wherein it further comprises an activation sensor (17).

6. The percutaneous neurostimulator (11) of claim 1, wherein it is powered by a battery (13).

7. The percutaneous neurostimulator (11) of claim 1, wherein it is powered by an accumulator.

8. The percutaneous neurostimulator (11) of claim 1, wherein it is powered by a piezoelectric generator.

9. The percutaneous neurostimulator (11) of claim 1, wherein it is not applied directly on the skin.

10. The percutaneous neurostimulator (11) of claim 9, wherein it is directly integrated within the sole (10) of a shoe, within a tailor-made arch support, within elastic cuffs.

11. The percutaneous neurostimulator (11) of claim 1, wherein it is applied directly on the skin.
